(19) United States
(12) Patent Application Publication
(10) Pub. No.: US 2002/0177579 A1
(43) Pub. Date: Nov. 28, 2002

(54) Extended release formulation of water-soluble drugs

(76) Inventors: Larry Augsburger, Severna Park, MD (US); John A. Bonck JR., Westminster, MD (US); Albert W. Brzeczko, Baltimore, MD (US)

Correspondence Address: MAX STUL OPPENHEIMER, P.O. Box 50, Stevenson, MD, 21153, US

(21) Appl. No.: 10/012,227

(22) Filed: Nov. 5, 2001

Related U.S. Application Data
(60) Provisional application No. 60/246,017, filed on Nov. 6, 2000.

(51) Int. Cl. A61K 31/663; B29B 9/00; A61K 31/404; A61K 31/46

(52) U.S. Cl. 514/102; 514/304; 514/415; 264/5

(57) ABSTRACT

A novel process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specific drug release profile.

This application is entitled to, and claims the benefit of, priority from U.S. Provisional Application Serial No. 60/246,017, filed Nov. 6, 2000.

### FIELD AND BACKGROUND OF THE INVENTION

1. Field of the Invention

The invention relates in general to pharmaceuticals and more specifically to extended release formulations of water-soluble pharmacophores, and to a process for manufacturing such formulations.

2. Background

The invention described and claimed herein comprises a novel process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specific drug release profile.

Extended release formulations of pharmacophores are useful: they facilitate maintaining a controlled level of the pharmacophore in a patient's system, and they improve patient compliance with dosing schedules by reducing the number of doses which the patient must remember to take.

It has, however, proved difficult to produce extended release formulations of water-soluble pharmacophores. Solubility normally predisposes a drug to rapid release from dosage forms, whereas extended release formulations generally must be designed to slow the release of at least a portion of the drug so as to keep pace with the rate at which the drug is being eliminated and/or detoxified by the patient's body.

In addition to Diltiazem (described in detail below), examples of useful pharmacophores which are water-soluble include the following:

| | Drug | | Max. | Fraction | Probable |
|---|---|---|---|---|---|
| Drug | Action | Solubility | Dose | Absorbed | BCS Class |
| Granisetron | Anti- | “Readily | 1 mg | ˜90% | Class I |
| HCI | nauseant | Soluble” | | | |
| (Kytril) | | | | | |
| Fosinopril | ACE | 100 | 20 mg | 36% | Class III |
| Sodium | Inhibitor | mg/mL | | | |
| (Monopril) | | | | | |
| Sumatriptan | Migraine | “Readily | 50 mg | 15% | Class III |
| Succinate | | Soluble” | | | |
| (Imitrex) | | | | | |
| Quinapril HCI | ACE | “Freely | 40 mg | 60% | Class III |
| (Accupril) | Inhibitor | Soluble” | | | |
| Benazepril | ACE | >100 | 40 mg | 37% | Class III |
| HCI | Inhibitor | mg/mL | | | |
| (Rotensin) | | | | | |
| Metoprolol | Anti- | “Freely | 190 mg | ˜90% | Class I |
| Succinate | hyper- | Soluble” | | | |
| (Toprol XL) | tensive | | | | |

Other water-soluble pharmacophores are listed and identified as such in standard references known to those of skill in the art; one such reference would be the Physician's Desk Reference, published periodically by Medical Economics Co, Montvale NJ. The BCS (Biopharmaceutical Classification System) classes are described in Amidon, Lennernas et. al., Pharm. Res., 7:80 (1995). By way of summary, compounds are broadly divided into Class I (High Solubility/High Permeability), Class II (Low Solubility/High Permeability) and Class III (High Solubility/Low Permeability); Class IV, although not used above, includes Low Solubility/Low Permeability compounds. In general, solubility is a measure of the volume required to dissolve the largest manufactured dose at its pH of minimum solubility in the physiological range (pH 1-8), and high solubility is generally considered to be less than 250 mL; permeability is based on the fraction absorbed, and greater than 90% is generally considered high permeability.

### SUMMARY OF THE INVENTION

The foregoing problems are overcome, and other advantages are provided by a novel process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specific drug release profile.

It is an object of the invention to provide a process for producing extended release drug formulations.

It is a further object of the invention to provide extended release formulations of known water-soluble pharmacophores.

A principal feature of the invention is the ability to produce extended release formulations of water-soluble pharmacophores.

Among the advantages of the invention are extending the benefits of extended release to water-soluble pharmacophores.

These and other objects, features and advantages which will be apparent from the discussion which follows are achieved, in accordance with the invention, by providing a novel process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specific drug release profile.

The various features of novelty which characterize the invention are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

FIG. 1 is a flow chart of the process of producing an extended release formulation of a water-soluble pharmacophore.

FIG. 2 is a chart of examples of candidate water-soluble drugs.

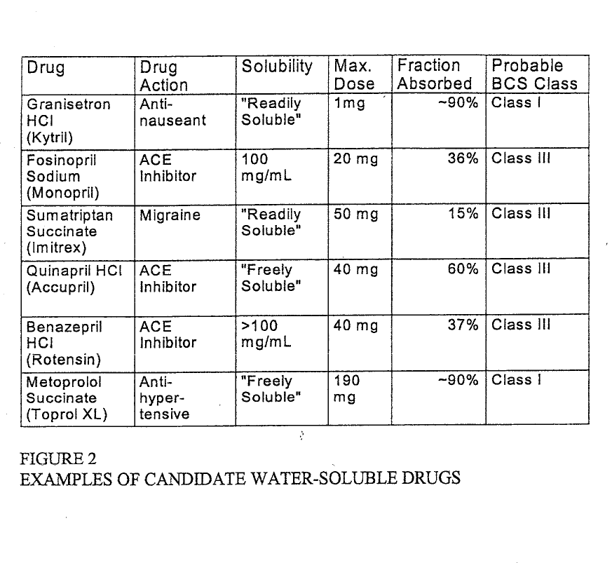

FIG. 3 is a flow chart for production of modified diltiazem hydrochloride powder.

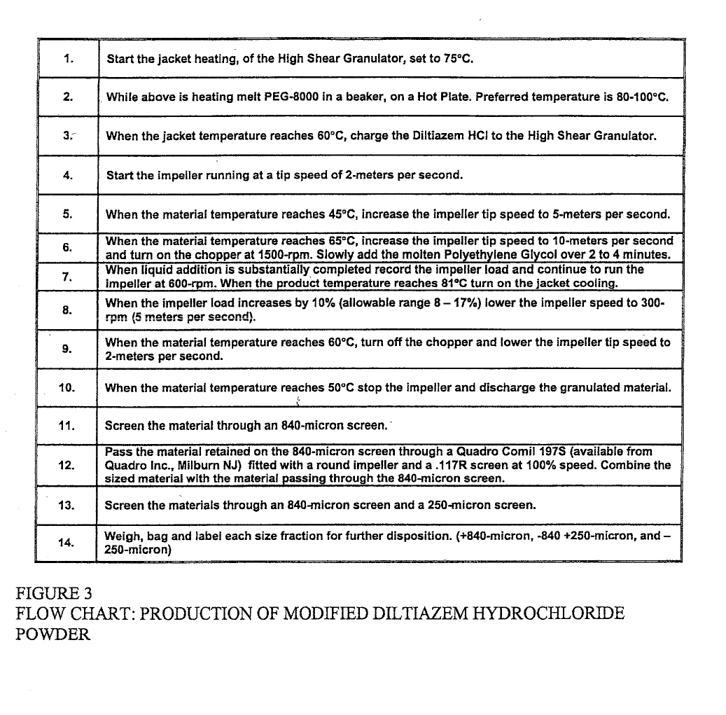

FIG. 4 is a chart showing an example calculation of particle mix.

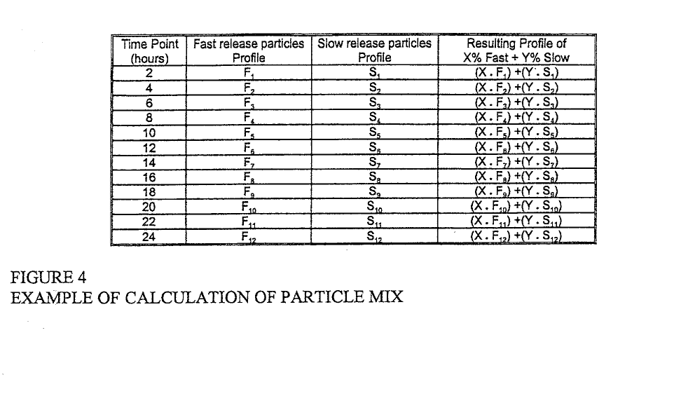

FIG. 5 is a flow chart for an example coating process.

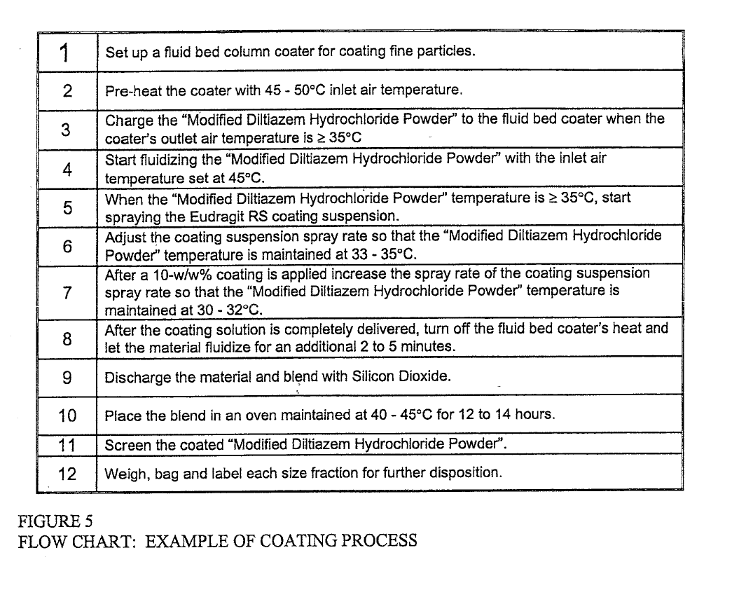

### DESCRIPTION OF THE PREFERRED EMBODIMENT

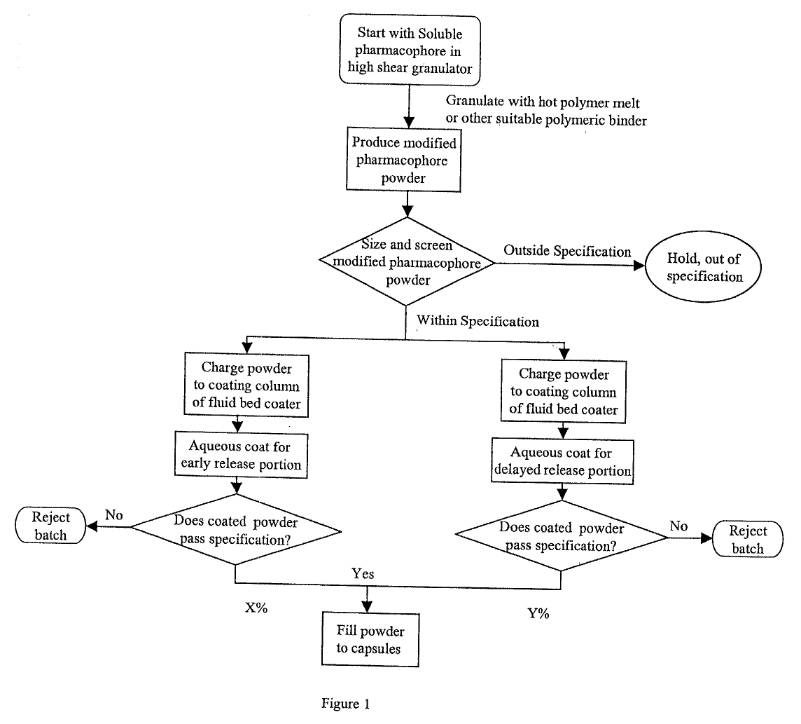

Referring to the drawings, the invention is a novel process for producing extended release formulation of a water-soluble drug comprising the steps of making a “modified drug” comprising a hot-melt granulation of the drug-with soluble or dispersible polymeric material of a suitable molecular weight and melting point, coating portions of the modified drug to different thicknesses or weights so as to produce subbatches, and blending the subbatches to achieve a specific drug release profile. A flow chart of the generalized process is shown in overview in FIG. 1.

The principal steps of the process comprise first selecting a water-soluble pharmacophore which it is desired to formulate as an extended release drug, then making a hot-melt granulation of the pharmacophore with suitable soluble or dispersible polymeric material of a suitable molecular weight and melting point, thereby producing a “Modified Drug”. The modified drug is then divided into portions (the number of portions being determined by the desired extended release profile), and each portion is coated with a suitable coating substance of differing thickness or weight, thereby creating subbatches which differ in the thickness or weight of the coating substance. These subbatches are then blended so as to achieve the desired drug release profile, and the resulting blend is then filled into a suitable delivery vehicle (for example, gelatin capsules). It is important to select a size distribution of the “Modified Drug” to minimize particle segregation of the coated particles (which would lead to poor content uniformity of the filled capsules).

In general, the goal is to use particles of the same size (although in practice it is usually impossible to achieve this goal); otherwise, large differences in particle size can lead to segregation according to size. Here, however, the goal is not to produce identical drug particle sizes since separate batches with different coating thicknesses are to be used. Therefore, the sizes of the drug particles need to be different for each batch, and are calculated so as to produce a uniform size of modified (coated) drug particles.

Experimental Results

In order to illustrate how the process may be implemented, the following experiment was carried out. Except where otherwise noted, temperatures, times and other parameters are subject to variation within limits known to those skilled in the art or obtainable by routine experimentation.

Step 1: Choose a Water-Soluble Pharmacophore.

The pharmacophore chosen was Diltiazem HCl. Diltiazem hydrochloride is a white to off-white crystalline powder of fine needles that is freely soluble in water, methanol, and chloroform. It is slightly soluble in 100% ethanol and it is not soluble in benzene. It has a melting point of 207.5-212° C. Diltiazem hydrochloride is a benzothiazepine calcium ion influx inhibitor (slow channel blocker or calcium channel antagonist). The chemical name of diltiazem is 1,5-benzothiazepin-4 (5H) one, 3-(acetyloxy)-5-[2-(dimethylamino) ethyl]-2,3-dihydro-2-(4-methoxyphenyl)-, monohydrochloride, (+)-cis-4. The molecular weight of diltiazem hydrochloride is 450.98. (Diltiazem HCl) inhibits the influx of calcium (Ca2+) ions during membrane depolarization of cardiac and vascular smooth muscle. Diltiazem is well absorbed from the gastrointestinal tract and is subject to an extensive first-pass effect, giving an absolute bioavailability (compared to intravenous administration) of about 40%. Diltiazem undergoes extensive metabolism in which only 2% to 4% of the unchanged drug appears in the urine. There are many suppliers and manufacturers of diltiazem hydrochloride. The manufacturer chosen for this experiment was Fermion of Finland, supplied through Interchem Corp. of New Jersey, U.S.A. The drug substance used in this product had the following particle size distribution. The analysis was done using an ATM Sonic Sifter with 5 grams using a pulse setting of 5 and a sift setting of 5.

| SCREEN MICRON | % RETAINED ON |
|---|---|
| OPENING | SCREEN |
| 212 | 41.4 |
| 180 | 1.1 |
| 150 | 1.6 |
| 125 | 2.0 |
| 90 | 3.6 |
| 53 | 6.6 |
| Pan | 43.7 |

Step 2: Choose the Desired Extended Release Profile

The profile selected was a 24-hour release similar to that of CARDIZEM CD. CARDIZEM® CD is manufactured by Hoechst Marion Roussel. The process for manufacturing CARDIZEM® CD involves layering diltiazem hydrochloride onto pareils. The drug layered spheres are then coated with a combination of polymers. The coated spheres are then filled into capsules. The compound and its formulation is described in more detail in the following US Patents, which are incorporated herein by reference: U.S. Pat. Nos. 5,616,345, 5,364,620, 5,002,776 and 4,894,240.

CARDIZEM® CD is formulated as a once-a-day extended release capsule containing either 120 mg, 180 mg, 240 mg, or 300-mg diltiazem hydrochloride. CARDIZEM® CD also contains: black iron oxide, ethyl-cellulose, FD&C Blue #1, fumaric acid, gelatin-NF, sucrose, starch, talc, titanium dioxide, white wax, and other ingredients. To achieve therapeutic blood levels of diltiazem hydrochloride the release rate of the compound must be controlled. Manufacturing and formulation methods for CARDIZEM® CD are covered under various patents (U.S. Pat. No. 5,002,776, 5,364,620, 5,439,689, 4,894,240, 5,470,584, & 5,286,497). In each patent, it is always mentioned that the diltiazem is delivered as a pellet or a core of diltiazem. It is also claimed that the pellet is made in association with an organic acid. Typically, the organic acid used in the formulation is Fumaric Acid. In the body of these patents are described methods of producing the diltiazem cores. The production of the cores is described as a layering process where diltiazem HCl is layered onto an inert core known as a non-pareil bead or seed of sugar/starch. Also covered is the coating of those cores. The patents always mention using organic solvents in the manufacturing of the CARDIZEM® CD product. Organic solvents are used in layering the diltiazem HCl onto the cores. Organic solvents are also used in coating the polymer onto the cores.

It should be noted that other patents describe alternative methods for the production of the diltiazem HCl core. The Andrx patent (U.S. Pat. No. 5,567,441) describes a layering process to produce the diltiazem HCl core. They claim they do not require a “stair-step release profile” (U.S. Pat. No. 5,286,497) to obtain a 24-hour release profile. The Biovail patent covers the manufacture of a diltiazem pellet using the extrusion/spheronization method. Here they claim that no organic solvents are needed to produce this bead. Beads manufactured by the extrusion/spheronization process are typically made by wetting the required ingredients in a suitable mixer or blender to produce a wet mass. The wet mass is then passed through a screen having a set screen size. An extruder is used to push the wet mass through the screen. If the formulation and moisture level of the mass are correct, the mass will form short extrudates. The extrudates are short rods, 25-mm to 75-mm in length. The extrudates are then placed into a bowl with a rotating disc on the bottom called a spheronizer. The spheronizer breaks the rods into beads. The bead size is directly related to the screen size used on the extruder. If a 1.0-mm diameter screen is used in the extruder then the average bead size produced by the spheronizer will be 1.0-mm. The length of time in the spheronizer and speed of the spheronizer's disc determines the uniformity and roundness of the beads. After the beads are produced, they are dried to obtain the desired final moisture.

Three lots of CARDIZEM CD had the following dissolution profile when measured in water.

(method: Apparatus 11, Paddle at 100-rpm, De-ionized water medium)

| Time | CARDIZEM CD | CARDIZEM CD | CARDIZEM CD |
|---|---|---|---|
| (hours) | Lot P90291 | Lot P90283 | Lot P90264 |
| 2 | 0.8 | 1.6 | 1.8 |
| 4 | 17.8 | 22.1 | 21.4 |
| 6 | 37.1 | 37.4 | 37.7 |
| 8 | 39.6 | 39.4 | 39.4 |
| 10 | 40.3 | 40.3 | 40.0 |
| 12 | 41.0 | 41.5 | 40.9 |
| 14 | 45.9 | 45.7 | 45.0 |
| 16 | 61.1 | 60.1 | 61.2 |
| 18 | 91.5 | 78.4 | 80.8 |
| 24 | 99.8 | 102.3 | 96.6 |

The dissolution profile of one of the lots of CARDIZEM CD was also measured in 2 other media (6.8 pH buffer and 0.1N HCl).

(method: Apparatus II, Paddle at 100-rpm, medium as shown below)

| | | CARDIZEM CD | CARDIZEM CD |
|---|---|---|---|
| Time | CARDIZEM CD | Lot 90264 | Lot 90264 |
| (hours) | Lot P90264 | in 6.8 pH buffer | in 0.1 N HCl |
| 2 | 1.8 | 0.8 | 0.8 |
| 4 | 21.4 | 24.1 | 8.0 |
| 6 | 37.7 | 33.4 | 28.9 |
| 8 | 39.4 | 34.0 | 31.5 |
| 10 | 40.0 | 34.5 | 31.6 |
| 12 | 40.9 | 36.5 | 31.7 |
| 14 | 45.0 | 46.3 | 31.9 |
| 16 | 61.2 | 64.0 | 33.3 |
| 18 | 80.8 | 75.5 | 42.8 |
| 24 | 96.6 | 82.3 | 73.3 |

Step 3: Make a Hot-Melt Granulation of the Pharmacophore with Suitable soluble or Dispersible Polymeric Material of a Suitable Molecular Weight and Melting Point, thereby Producing a “Modified Drug”.

The diltiazem hydrochloride was converted to “modified diltiazem hydrochloride powder” using molten Carbowax 8000 (TM) polyethylene glycol. It was found that two different coating levels of a methacrylate are required to ensure a controlled release of diltiazem hydrochloride from the modified drug particles over 24-hours. The particle size distribution of the coated modified particles was also found to influence the release rate of each coating level. The thickness (weight) of the coating on each of the two batches, the size distribution of the coated particles, and the ratio at which two different coated batches are mixed all were optimized to achieve a specified release profile. Optimization is achieved by first estimating the necessary parameters, then experimentally verifying that the result is satisfactory; if the result is not satisfactory, modifications can be made according to principles known to those skilled in the art, either empirically or with a more formal statistical approach. An example of the more formal approach would be to select independent variables such as (1) drug/PEG ratio, (2) modified powder particle size, (3) coating thickness, and (4) ratio of mixing of batches having two different coating thicknesses. Fixing the drug/PEG ratio would simplify the procedure. Possibly, the response could be a similarity metric, such as f2 (described below) that expresses how closely a profile is to another (reference) dissolution profile. A central composite responsesurface experimental design with f2 as the response could be constructed for the remaining independent variables, with each at three levels in the design.

As noted above, size distribution is important. The “Modified Diltiazem Hydrochloride Powder's” particle size distribution was chosen such that once it was coated, the larger, coated particles would not segregate from any smaller particles. The “Modified Diltiazem Hydrochloride Powder” used in the coating process was optimized to use particles passing through an 840-micron screen and retained on a 250-micron screen. A wide particle size distribution had an unacceptable content uniformity because of particle segregation.

The first step in the formulation development process was to achieve a dense, fine granular particle so that the powder would be free flowing. In addition, each particle should have a smooth surface. These two properties would yield a powder which would easily flow throw the coating system and have particles which would be easy to coat. Initial investigation of the diltiazem hydrochloride powder found it to have poor flow properties due to irregular particle surfaces. Since the diltiazem hydrochloride powder did not meet the needed criteria to be coated, the powder would first need to be modified. The goal was to produce a modified diltiazem hydrochloride powder consisting of small (150-840 microns in size) particles.

A High Shear Granulator (Model GP-110 liter Niro-Fielder, available from Niro Inc. Columbia, Md.) was used with either Eudragit NE 30D and/or hot wax as the powder modifying media. Both techniques produced acceptable modified diltiazem hydrochloride powder. However, the different techniques produced different dissolution profiles. A comparison of the dissolution profiles of the two different techniques is shown below. The two batches shown were coated to similar levels using the same coating polymer (Eudragit RS 30D). Their release characteristics were determined using a type 2 dissolution apparatus per current USP methods in water (37° C. at 100-rpm paddle speed).

| | Granulated with | Granulated with Hot Wax |
|---|---|---|
| Time | Eudragit NE 30D | (PEG 8000) |
| (hours) | (70% RS 30D coating) | (65% RS 30D coating) |
| 1 | 26.0% | |
| 2 | 66.0% | 5.0% |
| 3 | 88.0% | |
| 4 | 94.6% | 14.0% |
| 5 | 96.9% | |
| 6 | 100.0% | 28.7% |
| 7 | 100.0% | |
| 8 | 100.0% | 39.7% |

The modified diltiazem hydrochloride powder used for the 70% coating had 80% of the particle passing through a 590-micron screen and retained on a 125-micron screen, that used for the 65% coating had 100% of the particles passing through a 1190-micron screen and retained on a 177-micron screen. The coated diltiazem hydrochloride powder, for the 70% coating had 80% of the particles passing through an 840-micron screen and retained on a 250-micron screen, with mean particle size of 425 microns. The coated diltiazem hydrochloride powder for the 65% coating had 100% of the particles passing through a 1410-micron screen and retained on a 177-micron screen with mean particle size of 475 micron. Some of the difference in dissolution rate could have been caused by this particle size distribution difference. A dissolution result from a batch similar to the 65% coating was examined using only particles passing through a 420-micron screen and retained on a 300-micron screen with the following results:

| | | Granulated with Hot Wax |
|---|---|---|
| | | (PEG 8000) |
| | Granulated with | (65% RS 30D coating |
| Time | Eudragit NE 30D | with 100% particles |
| (hours) | (70% RS 30D coating) | −420 + 177-micron) |
| 1 | 26 0% | |
| 2 | 66.0% | 5.1% |
| 3 | 88.0% | |
| 4 | 94.6% | 12.1% |
| 5 | 96.9% | |
| 6 | 100.0% | 25.9% |
| 7 | 100.0% | |
| 8 | 100.0% | 65.5% |

The mean particle size was 360-microns for the 70% composition versus 425-microns for the 65% composition, yet the dissolution profile for the 65% composition was slower. It was concluded that a hot melt wax technique for producing the modified diltiazem hydrochloride powder yielded the best particle for coating. Polyethylene Glycol 8000 was chosen as the hot melt wax of choice. Polyethylene Glycol 8000 gave the highest bulk density of the powder (0.55 g/cc). After coating, batches made with PEG 8000 were found to have the slowest release. After powder modification, batches made with PEG 8000, were found to have the tightest particle size distribution.

It was also found that the modified diltiazem hydrochloride powder's particle size distribution had to be more appropriately defined to ensure a reproducible process. In the beginning of the development process, the powder to be coated used particles having sizes that pass through a 1190-micron screen but are retained on a 177-micron screen. This was too large of a particle size distribution. The initial wide particle size distribution caused segregation problems later in the capsule filling process. When segregation of particles occurred, the dissolution profile was not reproducible from one capsule to the next. In addition, when different coating levels were mixed in a single capsule, the resulting dissolution profile was not similar to the calculated dissolution profile. Having a tighter size distribution was found to avoid these problems. It was found that a starting powder having particles passing through an 840-micron screen and retained on a 250-micron screen yielded a controlled process. The coated powder could then be separated into particles passing through a 1000-micron screen and retained on a 250-micron screen.

Experimentally, the useable yield from the diltiazem hydrochloride powder modification technique was 60% to 75%. As this process is scaled up, this yield is anticipated to improve. Better hot liquid introduction systems are available on larger equipment. Processing methods will also improve the yield (main impeller speed, batch temperature at which to switch to cooling, sizing screen size, etc.).

The preferred method of production of the “modified diltiazem hydrochloride powder” is as follows:

| | |
|---|---|
| I. | Start the jacket heating, of the High Shear Granulator, set to 75° C. |
| II. | While above is heating melt PEG-8000 in a beaker, on a Hot Plate. Preferred temperature is 80- |
| | 100° C. |
| III | When the jacket temperature reaches 60° C., charge the Diltiazem HCl to the High Shear |
| | Granulator. |
| IV. | Start the impeller running at a tip speed of 2-meters per second. |
| V. | When the material temperature reaches 45° C., increase the impeller tip speed to 5-meters |
| | per second. |
| VI. | When the material temperature reaches 65° C., increase the impeller tip speed to 10-meters |
| | per second and turn on the chopper at 1500-rpm. Slowly add the molten Polyethylene Glycol |
| | over 2 to 4 minutes. |
| VII. | When liquid addition is substantially completed record the impeller load and continue to run |
| | the impeller at 600-rpm. When the product temperature reaches 81° C. turn on the jacket |
| | cooling. |
| VIII. | When the impeller load increases by 10% (allowable range 8-17%) lower the impeller speed |
| | to 300-rpm (5 meters per second). |
| IX. | When the material temperature reaches 60° C., turn off the chopper and lower the impeller tip |
| | speed to 2-meters per second. |
| X. | When the material temperature reaches 50° C. stop the impeller and discharge the granulated |
| | material. |
| XI. | Screen the material through an 840-micron screen. |
| XII. | Pass the material retained on the 840-micron screen through a Quadro Comil 197S (available |
| | from Quadro Inc., Milburn NJ) fitted with a round impeller and a .117R screen at 100% speed. |
| | Combine the sized material with the material passing through the 840-micron screen. |
| | Screen the materials through an 840-micron screen and a 250-micron screen. |
| | Weigh, bag and label each size fraction for further disposition. (+840-micron, −840 +250- |
| | micron, and −250-micron) |

The Polyethylene Glycol 8000 should be above its melting point of 65° C. before it touches the diltiazem hydrochloride powder. To ensure it is still above the melting point it is critical that the molten Polyethylene Glycol 8000 be at least 70° C. It is preferred that it be hofter. Temperatures at 100° C. are not unreasonable but are difficult to handle. If the molten Polyethylene Glycol 8000 is sprayed (not poured) onto the diltiazem hydrochloride powder a minimum temperature of 90° C. is preferred.

Creating the “Modified Diltiazem Hydrochloride Powder” requires that enough work input be used to increase the size of the granule; it was found that-additional work input would improve the yield. In addition, it was found that the molten Polyethylene Glycol 8000 was best added when the bed was greater than 65° C.

The heated “Modified Diltiazem Hydrochloride Powder” should not stop moving in the High Shear granulator until the powder's temperature is brought below the melting point of the Polyethylene Glycol 8000. It is therefore preferred that the bed only be discharged once the powder temperature is 55° C. or less.

The mill screen size is not critical in changing the activity of the diltiazem hydrochloride. The mill screen size will only affect the batch yield. The mill screen size may need to change as the product is scaled to different size and types of equipment.

Step 4: Divide the Modified Drug into Portions (The Number of Portions being Determined by the Desired Extended Release Profile), and Coat Each Portion with a Suitable Coating Substance of Differing Thickness or Weight, thereby Creating Subbatches which Differ in the Thickness or Weight of the Coating Substance.

The “modified diltiazem hydrochloride powder” was then coated with either a 55-w/w% or 75-w/w% of plasticized Eudragit RS and talc. These compositions may be selected empirically, selecting subbatches that exhibit dissolution profiles above and below the target, comparing the profile obtained with the selected subbatches against the target, then adjusting the ratios depending on the comparison.

Rohm America Polymers' Eudragit RS 30 D was found to offer the best solution for this application. Eudragit RS 30D is an Ammonio Methacrylate Copolymer, which is sparingly permeable. This polymer conforms to USP for “Ammonio Methacrylate Copolymer, Type B” and drug master file number 1242. It was chosen because of its low permeability in relationship to other polymers and the ability to adjust its film forming abilities. The Eudragit RS polymer is pH-independent so that the diltiazem hydrochloride releases slowly throughout the entire digestive tract. Finally, it was selected because it is an aqueous based coating system. This avoids the use of organic solvents that would present environmental problems and increase the costs associated with coating. Though it is an aqueous based coating system, solids concentrations may be as high as 25%.

It was applied as an aqueous based coating system. It was found that two different coating levels are required to ensure a controlled release of diltiazem hydrochloride over 24-hours while still having an immediate release portion after administration. A powder with a total coating level of 55-w/w% (21.3% polymer) was used for the early releasing portion of the formulation. A powder with a total coating level of 75-w/w% (31.9% polymer) was used for the delayed release portion of the formulation. The particle size cut of the coated powder was also found to influence the release rate of each coating level. The 55-w/w% coated powder that was used in the capsules, had particles that passed through a 1000-micron screen and were retained on a 250-micron screen. The 75-w/w% coated powder, which was used in the capsules, had particles that passed through a 1000-micron screen and were retained on a 590-micron screen. This particle size cut was used to achieve the slower release needed for the delayed portion of the formulation.

Since mean particle size of the modified diltiazem hydrochloride powder was small, the surface area per gram of powder was high. Therefore, the coating system needed to be efficient in restricting the diffusion of the drug out of the particle with a minimum amount of coating.

The formulation for the coating system was researched using different talc to polymer ratios and with increasing solution solid concentrations. The objective was to reduce the possibility of the diltiazem hydrochloride dissolving during the coating process and migrating into the coating. The coating manufacturer recommends talc to polymer solids levels of 0.5:1 to 1:1. The talc to polymer solids levels investigated were 0.25:1, 0.5:1, 0.75:1 and 1:1. Lower ratios produced coated particles that would stick and clump, and hence the powder would not flow easily into the coating column. Observing the flow of the modified diltiazem hydrochloride powder in the Fluid Bed coater (MP-1 Precision Coater Niro-Aeromatic, available from Niro Inc. Columbia, Md.) determined the level of Talc required. Flow of the coated powder into the coating column will progressively become less and less if there is not enough flow aid in the coating solution. This may be observed by watching the consistency of the flow in the down bed or by watching the bed pressure differential. Observing the down bed flow revealed that it would stop flowing at higher coating levels with talc levels of 0.5:1 or less. The bed differential pressure drop was also observed. As less powder flows through the coating column, the bed differential pressure drop will also drop off. With a talc level of 0.75:1 or less, the bed differential pressure drop would slowly drop off as the coating process proceeded. This could present a problem when the process is scaled into larger equipment and the bed loading is higher. With a talc level of 1:1, the bed pressure did not drop off with time. The 1:1 talc to polymer level was chosen because it allowed the coated powder to maintain sufficient flow properties throughout the coating process.

The coating system's solids concentration level was also investigated. Finding the maximum practical concentration of solids in the coating solution ensures an efficient coating process. With a higher concentration of solids, there is less chance of over-wetting the Diltiazem HCl particles. This is because a higher solids concentration permits a faster coating process since less water needs to be dried by the airflow through the coater. This allows the coating solution to be sprayed faster. The coating manufacturer recommends solids concentration levels from 20% to 25%. Solids concentration levels of 20%, 22.5% and 25% were tested. A 25% coating solution was found to work satisfactorily. However, it is not easy to handle and an experienced operator is required to ensure that the coater operates properly. Proper preparation, screening and handling of the solution is required. All batches were run using the coating manufacturer's guidelines for preparation and use of the coating solution.

Finally, the coating level needed to obtain the release profile required was examined. The particle size cuts for both the “Modified Diltiazem Hydrochloride Powder” and the “Coated Modified Diltiazem Hydrochloride Powder” was not tightly controlled in early development runs. Initial diltiazem hydrochloride runs were conducted using modified diltiazem hydrochloride powder with a particle size cut that passed through a 1190-micron screen and was retained on a 177-micron screen. The final coated powder (which was filled into capsules) had a particle size cut that passed through a 1410-micron screen and was retained on a 177-micron screen. Low coating level particles were mixed with high coating level particles so that the initial release would be fast but would still slowly release over 24-hours. The percentage to be used of fast releasing particles (low coating level) and slow releasing particles (high coating level) was based upon a calculation.

The calculation was as follows:

Calculation of Mixing fast and slow released particles
(Note: X% + Y% = 100%)

| | Fast release | Slow release | |
|---|---|---|---|
| Time Point | particles | particles | Resulting Profile of |
| (hours) | Profile | Profile | X% Fast + Y% Slow |
| 2 | F₁ | S₁ | (X * F₁) + (Y * S₁) |
| 4 | F₂ | S₂ | (X * F₂) + (Y * S₂) |
| 6 | F₃ | S₃ | (X * F₃) + (Y * S₃) |
| 8 | F₄ | S₄ | (X * F₄) + (Y * S₄) |
| 10 | F₅ | S₅ | (X * F₅) + (Y * S₅) |
| 12 | F₆ | S₆ | (X * F₆) + (Y * S₆) |
| 14 | F₇ | S₇ | (X * F₇) + (Y * S₇) |
| 16 | F₈ | S₈ | (X * F₈) + (Y * S₈) |
| 18 | F₉ | S₉ | (X * F₉) + (Y * S₉) |
| 20 | F₁₀ | S₁₀ | (X * F₁₀) + (Y * S₁₀) |
| 22 | F₁₁ | S₁₁ | (X * F₁₁) + (Y * S₁₁) |
| 24 | F₁₂ | S₁₂ | (X * F₁₂) + (Y * S₁₂) |

The preferred coating process for both the 55-w/w% and 75-w/w% coating is as follow:

| | |
|---|---|
| 1 | Set up a fluid bed column coater for coating fine particles. |
| 2 | Pre-heat the coater with 45-50° C. inlet air temperature. |
| 3 | Charge the “Modified Diltiazem Hydrochloride Powder” to the fluid bed coater when the |
| | coater's outlet air temperature is ≧ 35° C. |
| 4 | Start fluidizing the “Modified Diltiazem Hydrochloride Powder” with the inlet air |
| | temperature set at 45° C. |
| 5 | When the “Modified Diltiazem Hydrochloride Powder” temperature is ≧ 35° C., start |
| | spraying the Eudragit RS coating suspension. |
| 6 | Adjust the coating suspension spray rate so that the “Modified Diltiazem Hydrochloride |
| | Powder” temperature is maintained at 33-35° C. |
| 7 | After a 10-w/w % coating is applied increase the spray rate of the coating suspension |
| | spray rate so that the “Modified Diltiazem Hydrochloride Powder” temperature is |
| | maintained at 30-32° C. |
| 8 | After the coating solution is completely delivered, turn off the fluid bed coater's heat and |
| | let the material fluidize for an additional 2 to 5 minutes. |
| 9 | Discharge the material and blend with Silicon Dioxide. |
| 10 | Place the blend in an oven maintained at 40-45° C. for 12 to 14 hours. |
| 11 | Screen the coated “Modified Diltiazem Hydrochloride Powder”. |
| 12 | Weigh, bag and label each size fraction for further disposition. |

The starting “Modified Diltiazem Hydrochloride Powder” used in the coating process needs to be in a specific starting size range to ensure that the dissolution of the coated material will have a repeatable release profile. It was found that the “Modified Diltiazem Hydrochloride Powder” worked best when the starting particles passed through an 840-micron screen and were retained on a 250-micron screen.

The fluid bed operating parameters used for applying coating were standard operating conditions recommended by the polymer supplier. However, it is critical to hold to these conditions. The inlet air temperature should be maintained at 40 to 50° C. The bed temperature should be maintained at 28-35° C. during the run. If these temperatures are exceeded on the lower end there is the chance of sticking particles together because they are too wet. If the temperatures are exceeded on the upper end there is the risk of sticking particles together because the polymer is too hot. In the beginning of the coating process it is preferred that the bed temperature be held at 33-35° C. although this was not shown to be critical. The amount of airflow through the coater depends upon the size of the column being used. The velocity through the column should be in the range of 5 to 7 m/sec. On a smaller unit it is preferred that the air velocity through the column be on the lower end so the particles do not impinge on the filters.

After the powder is coated it should be blended with Colloidal Silicon Dioxide or some other suitable material to prevent the coated powder from sticking together and removing the coating. The coating was stabilized by holding the powder at 40 -45° C. for at least 12 hours. Longer times will not harm the coating.

Finally, sizing the coated powder is critical to achieve a repeatable dissolution profile. The 55-w/w% coated powder used particles that passed through a 1000-micron screen and were retained on a 250-micron screen. The 75-w/w% coated powder used particles that passed through a 1000-micron screen and were retained on a 590-micron screen.

Step 5: Blend the Subbatches so as to Achieve the Desired Drug Release Profile

A desired dissolution profile was developed by using the release profile of each coating level. The percentage of each coating level was changed until the calculated release profile gave the desired results. When the actual product was first tested using the calculated percentages, the actual dissolution profile did not match the calculated profile. Further investigation found that the particle size cut used for coating was too wide. Once the particle size cuts were controlled to a tighter defined range, the calculated dissolution profile matched the actual dissolution profile. The particle size cut for the “Modified Diltiazem Hydrochloride Powder” that was used for coating was therefore narrowed to a size cut that passed through a 1000-micron screen and was retained on a 250-micron screen. Different coating levels were then applied to determine which coating level would yield the best results. For the higher coating levels the dissolution of various particle size ranges was also examined with the following results.

Release Profiles (%) for coating levels and particle sizes
of “Modified Diltiazem Hydrochloride Powder”
(method: Apparatus II, Paddle at 100-rpm, De-ionized water medium)

| | 60% | 65% | 70% | 70% | 75% | 75% |
|---|---|---|---|---|---|---|
| Coating Level | −1000 | −1000 | −1000 | −1000 | −1000 | −1000 |
| Size | +250 | +250 | +300 | +420 | +300 | +420 |
| Distribution | microns | microns | microns | microns | microns | microns |
| 2 | 16.9 | 13.2 | 5.2 | 4.0 | 1.1 | 5.1 |
| 4 | 45.0 | 42.1 | 28.6 | 7.6 | 18.7 | 6.5 |
| 6 | 65.4 | 62.7 | 68.4 | 11.0 | 50.9 | 4.5 |
| 8 | 79.5 | 75.2 | 92.8 | 28.8 | 94.7 | 13.0 |
| 10 | 89.2 | 85.0 | 96.2 | 46.9 | 100.0 | 23.5 |
| 12 | 95.2 | 91.9 | 96.7 | 60.7 | 100.0 | 35.4 |
| 14 | 98.1 | 96.3 | 99.5 | 71.2 | 100.0 | 49.9 |
| 16 | 100.0 | 98.9 | 100.0 | 81.0 | 100.0 | 60.5 |
| 18 | 100.0 | 100.0 | 100.0 | 89.7 | 100.0 | 71.6 |
| 20 | 100.0 | 100.0 | 100.0 | 92.2 | 100.0 | 78.9 |
| 22 | 100.0 | 100.0 | 100.0 | 98.2 | 100.0 | 91.5 |
| 24 | 100.0 | 100.0 | 100.0 | 100.0 | 100.0 | 97.3 |

Calculated Versus Actual release Profiles for blends
of coated “Modified Diltiazem Hydrochloride Powder”
method: Apparatus II, Paddle at 100-rpm, De-ionized water medium)

| | Calc | Actual | Calc | Actual |
|---|---|---|---|---|
| Time | 30% of 55% | 30% of 55% | 35% of 60% | 35% of 60% |
| (hours) | 70% of 75% | 70% of 75% | 65% of 75% | 65% of 75% |
| 2 | 13.1 | 11.8 | 7.3 | 6.2 |
| 4 | 23.6 | 21.6 | 15.7 | 13.1 |
| 6 | 29.1 | 26.7 | 21.2 | 19.6 |
| 8 | 32.5 | 30.2 | 25.6 | 24.4 |
| 10 | 34.8 | 32.2 | 32.5 | 28.3 |
| 12 | 37.9 | 35.1 | 38.8 | 33.4 |
| 14 | 45.0 | 41.7 | 48.9 | 41.5 |
| 16 | 57.0 | 53.0 | 60.0 | 54.7 |
| 18 | 70.3 | 66.6 | 72.4 | 68.0 |
| 20 | 82.1 | 78.9 | 83.4 | 79.5 |
| 22 | 92.5 | 90.1 | 93.0 | 89.7 |
| 24 | 98.5 | 99.6 | 98.6 | 98.4 |

Step 6: Fill a Suitable Delivery Vehicle with the Resulting Blend.

Finally 30% of the 55-w/w% coated material plus 70% of the 75-w/w% coated material were filled to a capsule, preferably a hard gelatin capsule such as is available from the Capsugel Division of Pfizer, Inc. or Shionogi Qualicaps.

The resulting product was compared with the desired release profile by comparing dissolution profiles in 0.1N HCl.

The similarity factor as defined by f2 was used to determine whether two dissolution profiles are similar:

f₂=50 LOG {[1+1/NΣ^{N}_{T=1}(R_{T}−T_{T})²]^{0.5}×100}

Rₜ and Tₜ are the percent dissolved at each time point for two different dissolution profiles. A f₂ between 50 and 100 suggests the two dissolution profiles are similar. The f₂ factor is considered conservative. While two dissolutions, which do not meet the f₂, criteria may still be equivalent when tested in-vivo it is unlikely that two similar dissolution profiles will be non-equivalent when tested in-vivo.

The f₂ value comparing the experimental product to CARDIZEM® CD was 56.8.

The f₂'s for the dissolution curves “calculated” versus “actual” were 76.9 for the 30% of 55-w/w% coat plus 70% of 75-w/w% coating and 69.5 for the 35% of 60-w/w% coat plus 65% of 75-w/w% coating. The profile that was chosen was 30% of 55-w/w% coat plus 70% of 75-w/w% coating because it had an initial fast release followed by a constant rate release.

The preferred coating level was determined by obtaining a dissolution profile that would release at a relatively constant rate over 24 hours. The experimental product was then compared to other diltiazem hydrochloride products on the market. CARDIZEM® CD and APO-DILTIAZ CD are reported to be bio-equivalent to each other, yet their dissolution profiles in water are quite dissimilar. The f₂ value comparing the CARDIZEM® CD and APO-DILTIAZ CD dissolution curves is 37.3. This dissimilarity demonstrates that in-vitro testing may not be predictive of in-vivo results because there are mechanistic differences in drug release for each product. The purpose of this comparison was to ascertain coating levels for 24-hour release in the experimental product. The dissolution profile for the experimental product was compared to each product's profile. The f₂ values comparing the experimental product's dissolution curve versus CARDIZEM® CD's and APO-DILTIAZ CD's dissolution curves in water are 55.0 and 42.9 respectively. A table of this data follows.

### Comparison of Release Profiles of different manufacturers of “Extended Release Diltiazem Hydrochloride Capsules”

(method: Apparatus II, Paddle at 100-rpm, De-lonized water medium)

| | | ApoTex | Blend of |
|---|---|---|---|
| Time | CARDIZEM CD | UPM Data | 30% 55% coat & |
| (hours) | Lot P90264 | Lot CA 990 | 70% 75% coat |
| 2 | 1.8 | 2.80 | 7.4 |
| 4 | 21.4 | 9.60 | 18.0 |
| 6 | 37.7 | 20.22 | 29.8 |
| 8 | 39.4 | 30.68 | 35.3 |
| 10 | 40.0 | 38.47 | 38.0 |
| 12 | 40.9 | 42.15 | 40.9 |
| 14 | 45.0 | 46.98 | 44.4 |
| 16 | 61.2 | 51.22 | 52.2 |
| 18 | 80.8 | 55.37 | 63.4 |
| 20 | | 60.88 | 76.4 |
| 22 | | 65.21 | 89.4 |
| 24 | 96.6 | 70.88 | 100.1 |

The intermediate modified diltiazem hydrochloride powder had the following formula.

| | |
|---|---|
| COMPONENT | Composition per tablet |

| (Trade Name) | mg per capsule | % per capsule |
|---|---|---|
| Diltiazem Hydrochloride | 120.00 | 85.00% |
| CARBOWAX 8000 | 21.18 | 15.00% |
| TOTAL | 141.18 | 100.00% |

The Diltiazem Hydrochloride Extended-release,
120-mg capsule formula WAS:

| | |
|---|---|
| COMPONENT | Composition per capsule |

| (Trade Name) | mg per capsule | % per capsule |
|---|---|---|
| Diltiazem Hydrochloride | 120.00 | 28.70% |
| CARBOWAX 8000 | 21.18 | 5.07% |
| EUDRAGIT RS | 124.92 | 29.88% |
| TRI-ETHYL CITRATE | 24.98 | 5.97% |
| TALC | 124.92 | 29.88% |
| Silicon Dioxide | 2.09 | 0.50% |
| TOTAL: | 418.09 | 100.00% |

The sources of the materials and their functions follow:

| DRUG PRODUCT | | |
|---|---|---|
| INGREDIENT/FUNCTION | trade name | manufacturer |
| Diltiazem Hydrochloride | Dilitiazem Hydrochloride | Fermion of Finland |
| (pharmacophore) | | supplied through |
| | | Interchem Corp. |
| | | New Jersey, U.S.A. |
| POLYETHYLENE GLYCOL 8000 | Carbowax 8000 | Union Carbide Corp. |
| (USED TO INCREASE PARTICLE | | Danbury, CT |
| SIZE OF PHARMACOPHORE WHILE | | |
| PROVIDING INSOLUBLE LAYER | | |
| FOR COATING) | | |
| AMMONIO METHACRYLATE | Eudragit RS | Rohm America Polymers |
| COPOLYMER, TYPE B | | Somerville, NJ |
| (FILM-FORMING POLYMER TO | | |
| DELAY RELEASE OF | | |
| PHARMACOPHORE) | | |
| TRI-ETHYL CITRATE | Tri-ethyl Citrate | Morflex, Inc. |
| (MODIFIES POLYMER'S FILM- | | Greensboro, NC |
| FORMING PROPERTIES) | | |
| TALC 140 BC | Talc | Whittaker Clark & Daniels, Inc. |
| (ANTI-STICKING AID FOR USE WITH | | South Plainfield, NJ |
| POLYMER COATING SUSPENSION) | | |
| COLLOIDAL SILICON DIOXIDE | Cab-O-Sil Untreated Fumed | Cabot Corporation |
| (FLOW AID AND ANTI-STICKING | Silica | Naperville, Il |
| AID, USED AFTER POLYMER IS | | |
| APPLIED) | | |

Bulk Densities

| PRODUCT | DENSITY | |
|---|---|---|
| 55-w/w % coated powder | 0.793 | g/cc |
| 75-w/w % coated powder | 0.853 | g/cc |
| Density of powder in Diltiazem Hydrochloride | 0.84 | g/cc |
| Extended-release Capsules | | |

Product Capsule sizes

| PRODUCT | CAPSULE SIZE |
|---|---|
| 120-mg | Size 1 or Size 1 ELO |
| 180-mg | Size 0 ELO |
| 240-mg | Size 00 |
| 300-mg | Size 000 |

The experimental drug product's dissolution was tested in 3 media.

(method: Apparatus II, Paddle at 100-rpm, medium as shown below)

| Time | | | |
|---|---|---|---|
| (hours) | Purified Water | in pH 6.8 buffer | in 0.1 N HCl |
| 2 | 7.4 | 15.3 | 11.7 |
| 4 | 18.0 | 25.4 | 23.8 |
| 6 | 29.8 | 42.8 | 28.4 |
| 8 | 35.3 | 61.6 | 30.6 |
| 10 | 38.0 | 66.8 | 33.2 |
| 12 | 40.9 | 66.8 | 35.0 |
| 14 | 44.4 | 66.8 | 38.1 |
| 16 | 52.2 | 68.3 | 42.8 |
| 18 | 63.4 | 68.2 | 53.4 |
| 20 | 76.4 | 68.2 | 61.0 |
| 22 | 89.4 | 68.7 | 71.8 |
| 24 | 100.1 | 69.2 | 83.2 |

Stability

To determine the stability of the product the capsules were placed in 40° C. and 75%RH (accelerated conditions) for 1 month after which time the dissolution profile was checked. Below is a comparison between the time zero profile and the 1-month profile.

(method: Apparatus II, Paddle at 100-rpm, De-ionized water medium)

| Time | | 4 week stability |
|---|---|---|
| (hours) | Time = 0 | @40 C/75% RH |
| 2 | 7.4 | 5.7 |
| 4 | 18.0 | 15.6 |
| 6 | 29.8 | 23.1 |
| 8 | 35.3 | 26.0 |
| 10 | 38.0 | 28.0 |
| 12 | 40.9 | 32.4 |
| 14 | 44.4 | 39.7 |
| 16 | 52.2 | 51.8 |
| 18 | 63.4 | 65.0 |
| 20 | 76.4 | 78.4 |
| 22 | 89.4 | 87.8 |
| 24 | 100.1 | 99.4 |

Dissolution Comparison in Different Media Comparison in Water (f₂=56.8)

(method: Apparatus II, Paddle at 100-rpm, De-ionized water medium)

| | | Blend of |
|---|---|---|
| Time | CARDIZEM CD | 30% 55% coat & |
| (hours) | Lot P90264 | 70% 75% coat |
| 2 | 1.8 | 7.4 |
| 4 | 21.4 | 18.0 |
| 6 | 37.7 | 29.8 |
| 8 | 39.4 | 35.3 |
| 10 | 40.0 | 38.0 |
| 12 | 40.9 | 40.9 |
| 14 | 45.0 | 44.4 |
| 16 | 61.2 | 52.2 |
| 18 | 80.8 | 63.4 |
| 20 | | 76.4 |
| 22 | | 89.4 |
| 24 | 96.6 | 100.1 |

Comparison in 0.1 N HCL: (f₂ = 53.4)
(method: Apparatus II, Paddle at 100-rpm, 0.1 N HCl medium)

| | CARDIZEM CD | Experimental |
|---|---|---|
| Time | Lot 90264 | blend |
| (hours) | in 0.1 N HCl | in 0.1 N HCl |
| 2 | 0.8 | 11.7 |
| 4 | 8.0 | 23.8 |
| 6 | 28.9 | 28.4 |
| 8 | 31.5 | 30.6 |
| 10 | 31.6 | 33.2 |
| 12 | 31.7 | 35.0 |
| 14 | 31.9 | 38.1 |
| 16 | 33.3 | 42.8 |
| 18 | 42.8 | 53.4 |
| 20 | | 61.0 |
| 22 | | 71.8 |
| 24 | 73.3 | 83.2 |

Comparison in 6.8 pH buffer: (f₂=35.8)

(method: Apparatus II, Paddle at 100-rpm, pH 6.8 buffer medium)

| | CARDIZEM CD | Experimental |
|---|---|---|
| Time | Lot 90264 | blend |
| (hours) | in 6.8 pH buffer | in pH 6.8 buffer |
| 2 | 0.8 | 15.3 |
| 4 | 24.1 | 25.4 |
| 6 | 33.4 | 42.8 |
| 8 | 34.0 | 61.6 |
| 10 | 34.5 | 66.8 |
| 12 | 36.5 | 66.8 |
| 14 | 46.3 | 66.8 |
| 16 | 64.0 | 68.3 |
| 18 | 75.5 | 68.2 |
| 20 | | 68.2 |
| 22 | | 68.7 |
| 24 | 82.3 | 69.2 |

Thus, there has been described a novel process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specific drug release profile. that has a number of novel features and advantages, and a manner of making and using the invention.

While a specific embodiment of the invention has been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure. Thus, the invention is not limited to the specific embodiment described herein, but is defined by the appended claims.

We claim:

1. A process for producing extended release formulation of a water-soluble drug comprising the steps of making a “modified drug” comprising a hot-melt granulation of the drug with soluble or dispersible polymeric material of a suitable molecular weight and melting point, coating portions of the modified drug to different thicknesses or weights so as to produce subbatches, and blending the subbatches to achieve a specified drug release profile.

2. An extended release drug comprising a granulation of a water soluble pharmacophore coated with a suitable coating material so as to produce granules of a modified drug, wherein there are at least two types of granules of the modified drug, said types distinguished by the thickness or weight of said coating material.

3. An extended release drug as in claim 2 wherein said pharmacophore is granisetron HCl.

4. An extended release drug as in claim 2 wherein said pharmacophore is fosinopril sodium.

5. An extended release drug as in claim 2 wherein said pharmacophore is sumatriptan succinate.

6. An extended release drug as in claim 2 wherein said pharmacophore is quinapril HCl.

7. An extended release drug as in claim 2 wherein said pharmacophore is benazepril HCl.

8. An extended release drug as in claim 2 wherein said pharmacophore is metoprolol succinate.
